(19) United States
(12) Patent Application Publication
(10) Pub. No.: US 2026/0033747 A1
(43) Pub. Date: Feb. 5, 2026

(54) MEMBRANE FOR PLATEN OF OPTICAL BIOMETRIC SENSOR

(71) Applicant: HID Global Corp., Austin, TX (US)

(72) Inventor: George William McClurg, Jensen Beach, FL (US)

(21) Appl. No.: 18/788,549

(22) Filed: Jul. 30, 2024

(51) Int. Cl. A61B 5/1172; A61B 5/00; B32B 7/06; B32B 27/36

(52) U.S. Cl. CPC A61B 5/1172; A61B 5/0064; B32B 7/06; B32B 27/365; B32B 2307/412; B32B 2535/00

(57) ABSTRACT

A user can removably adhere a transparent layer of a membrane to a platen of an optical biometric sensor. The membrane can include a transparent layer disposed between a protective cover sheet layer and a stiffener layer. The user can peel the protective cover sheet layer from the transparent layer to expose a first side of the transparent layer. The user can position the first side of the transparent layer against the platen such that the transparent layer is disposed between the platen and the stiffener layer. The transparent layer can adhere to the platen via optical contact bonding. The user can peel the stiffener layer from the transparent layer after the transparent layer has been adhered to the platen to expose a second side of the transparent layer. The stiffener layer and the protective cover sheet layer can optionally be similar or identical in material and structure.

### FIELD OF THE DISCLOSURE

This document relates generally to optical biometric sensors, such as for acquiring an image of a scannable portion of an anatomy, such as a fingerprint or a palmprint, such as for identification, enrollment, and/or verification.

### BACKGROUND OF THE DISCLOSURE

Biometric identification systems, such as fingerprint and palmprint identification systems, can identify, enroll, and/or verify an individual, such as for gaining access to a virtual or physical secured area, gaining access to a bank account, gaining access to a room or building through the unlocking of a door, and the like. Biometric identification systems can also be used for forensic identification, or other suitable applications. There is ongoing effort to improve biometric identification systems.

### SUMMARY

In an example, a membrane for a platen of an optical biometric sensor can include a protective cover sheet layer. The membrane can include a transparent layer removably contacting the protective cover sheet layer and can adhere, via optical contact bonding, to the platen after the protective cover sheet layer has been removed. The membrane can include a stiffener layer removably contacting the transparent layer such that the stiffener layer and the protective cover sheet layer are disposed on opposite sides of the transparent layer. The stiffener layer can be removable from the transparent layer after the transparent layer has been adhered, via optical contact bonding, to the platen.

In an example, a method can removably adhere a transparent layer of a membrane to a platen of an optical biometric sensor. The membrane can include the transparent layer disposed between a protective cover sheet layer and a stiffener layer. The method can include peeling the protective cover sheet layer from the transparent layer to expose a first side of the transparent layer. The method can include positioning the first side of the transparent layer against the platen such that the transparent layer is disposed between the platen and the stiffener layer. The method can include adhering the transparent layer to the platen via optical contact bonding. The method can include peeling the stiffener layer from the transparent layer after the transparent layer has been adhered to the platen to expose a second side, opposite the first side, of the transparent layer.

In an example, a membrane for a platen of an optical biometric sensor can include a protective cover sheet layer having a surface area that corresponds to a surface area of the platen. The membrane can include a transparent layer having a first side that removably contacts the protective cover sheet layer such that the protective cover sheet layer can be peeled off the transparent layer. The first side of the transparent layer can adhere, via optical contact bonding, to the platen after the protective cover sheet layer has been peeled off the transparent layer by being placed in contact with the platen. The transparent layer can be softer than the protective cover sheet layer. The transparent layer can have a surface area that corresponds to the surface area of the platen. The transparent layer can have a second side opposite the first side of the transparent layer. The membrane can include a stiffener layer having a first side that removably contacts the second side of the transparent layer. The stiffener layer can be stiffer than the transparent layer. The stiffener layer and the protective cover sheet layer can be formed from a same material, can have a same thickness, can have a same stiffness, and can have a same surface area that corresponds to the surface area of the platen. The stiffener layer can be peeled off the transparent layer after the transparent layer has been adhered to the platen to expose the second side of the transparent layering.

### BRIEF DESCRIPTION OF THE DRAWINGS

FIG. 1 shows a side-view schematic drawing of an example of a membrane for a platen of an optical biometric sensor.

FIG. 2 shows a side-view schematic drawing of a portion of the membrane of FIG. 1, after the protective cover sheet layer has been peeled off the transparent layer.

FIG. 3 shows a side-view schematic drawing of a portion of the membrane of FIG. 1, as the transparent layer has been placed in contact with the platen of the optical biometric sensor to adhere the transparent layer to the platen.

FIG. 4 shows a side-view schematic drawing of a portion of the membrane of FIG. 1, after the protective cover sheet layer has been peeled off the transparent layer, after the transparent layer has been adhered to the platen, and after the stiffener layer has been peeled off the transparent layer.

FIG. 5 shows a side-view schematic drawing of an example of an optical biometric sensor during use, in which the transparent layer has been adhered to the platen.

FIG. 6 shows a perspective-view drawing of an assembly apparatus for assembling the membrane of FIG. 1.

FIG. 7 shows a flow chart of an example of a method for removably adhering a transparent layer of a membrane to a platen of an optical biometric sensor.

### DETAILED DESCRIPTION

To identify a person, an optical biometric sensor can require the person to place a finger or palm against a platen, formed from glass, fused silica, plastic, or other suitable optical material. The optical biometric sensor can acquire one or more images of the fingerprint or palm print while the finger or palm is in contact with the platen. After the person has removed the finger or palm from the platen, the platen may retain all or a portion of the fingerprint or palm print, so that the retained portion may appear in subsequent images acquired by the optical biometric sensor. The retained portion may be referred to as a latent print image.

To reduce or eliminate latent print images, the optical biometric sensor may use a transparent layer, such as formed from silicone, disposed on the platen. The person can place a finger or palm on the transparent layer, such that during contact, when the optical biometric sensor is acquiring an image of the fingerprint or palm print, the transparent layer is disposed between the finger or palm and the platen. Such a transparent layer may be designed so as not to retain the fingerprint or palm print and may therefore reduce or eliminate the effect of latent print images. In addition, such a transparent layer may improve intimate contact between the finger or palm and the platen, which can improve the image quality. For the purposes of this document, the term transparent is intended to designate that an optical element or interface transmits at least eighty percent of the incident optical power through the optical element or interface.

To achieve high-quality imaging of the fingerprint or palm print, the transparent layer can be relatively thin. Such a thin transparent layer may be fragile, and therefore may be difficult to handle and place correctly on the platen. The membrane described in detail below can help deliver the transparent layer to the platen. The membrane can have an improved structural integrity, so that the membrane can withstand relatively rough handling and placement. The membrane can include removable elements, which can provide structural integrity to the transparent layer.

In an example, a user can removably adhere a transparent layer of a membrane to a platen of an optical biometric sensor. The membrane can include a transparent layer disposed between a protective cover sheet layer and a stiffener layer. The user can peel the protective cover sheet layer from the transparent layer to expose a first side of the transparent layer. The user can position the first side of the transparent layer against the platen such that the transparent layer is disposed between the platen and the stiffener layer. The transparent layer can adhere to the platen via optical contact bonding. Optical contact bonding can be a glueless process where two closely conformal surfaces (such as flat surfaces) are joined, such as being held together purely by intermolecular forces. The surfaces may be free from contamination, such as specks of dust. The surfaces may form the bond simply by being brought together without any pressure being applied. The intermolecular forces draw the surfaces into a lowest energy conformation. The user can peel the stiffener layer from the transparent layer after the transparent layer has been adhered to the platen to expose a second side of the transparent layer. The stiffener layer and the protective cover sheet layer can optionally be similar or identical in material and structure.

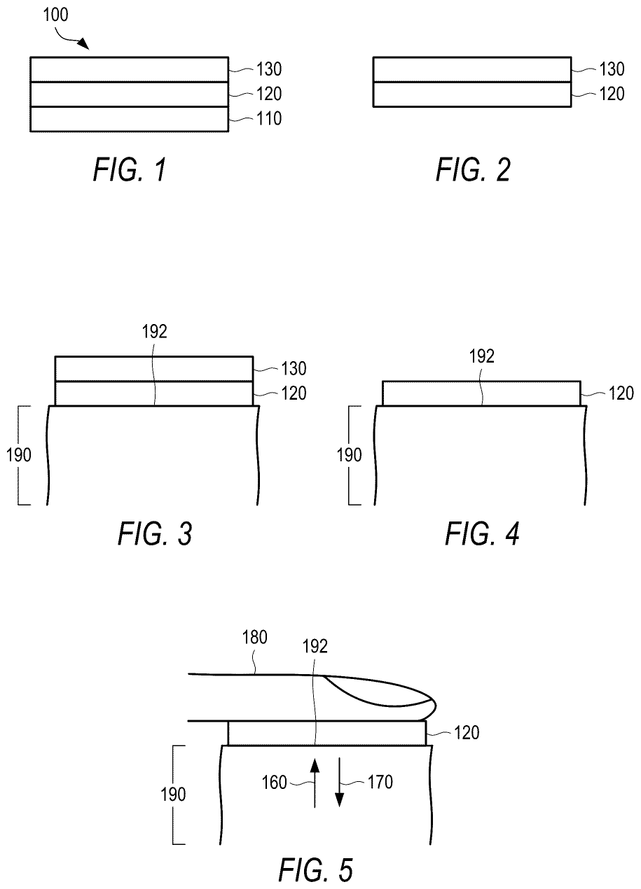

FIG. 1 shows a side-view schematic drawing of an example of a membrane 100 for a platen 192 (FIG. 5) of an optical biometric sensor 190 (FIG. 5). The membrane 100 can include a transparent layer 120 disposed between a protective cover sheet layer 110 and a stiffener layer 130. The configuration of FIG. 1 is but one example of a membrane 100 for a platen 192 of an optical biometric sensor 190; other suitable configurations can also be used.

The membrane 100 can include a protective cover sheet layer 110. The protective cover sheet layer 110 can protect the transparent layer 120 during the manufacturing and shipping processes for the membrane 100. The protective cover sheet layer 110 can be transparent, semi-transparent, or opaque. The protective cover sheet layer 110 can be formed from a clear polycarbonate film, or other suitable material.

The protective cover sheet layer 110 can have a surface area that corresponds to a surface area of the platen 192. For example, the membrane 100, in its assembled state, can have a surface area that is slightly smaller than a surface area of platen 192, such that a user can easily position the various layers of the membrane 100 on the platen 192 with relatively loose tolerances. After placement with the relatively loose tolerances, the various layers of the membrane 100 can have a specified orientation with respect to the platen 192. After placement with the relatively loose tolerances, the various layers of the membrane 100 can extend over a central area of the platen 192, such that one or more edges of the platen 192 may have one or more relatively small areas that remain uncovered by the various layers of the membrane 100. Alternatively, the membrane 100, in its assembled state, can have the same size and/or shape as the platen 192 or can even be larger than the platen 192. The protective cover sheet layer 110 can optionally include one or more tabs that extend outward beyond transparent layer 120 and the stiffener layer 130. The one or more tabs may be grippable, to allow a user to easily grip and remove the protective cover sheet layer 110 from the transparent layer 120.

The membrane 100 can include a transparent layer 120 removably contacting the protective cover sheet layer 110. In some examples, the transparent layer 120 and the protective cover sheet layer 110 may be held together by surface tension, such as without using an additional adhesive material between the transparent layer 120 and the protective cover sheet layer 110. In other examples, an adhesive, such as a relatively weak adhesive, can hold together the transparent layer 120 and the protective cover sheet layer 110. The membrane 100 may optionally include a release agent disposed between the transparent layer 120 and the protective cover sheet layer 110. The transparent layer 120 can adhere, via optical contact bonding, to the platen 192 after the protective cover sheet layer 110 has been removed. For example, the transparent layer 120 can have a first side that removably contacts the protective cover sheet layer 110 such that the protective cover sheet layer 110 can be peeled off the transparent layer 120. The first side of the transparent layer 120 can adhere, via optical contact bonding, to the platen 192 (FIG. 3) after the protective cover sheet layer 110 has been peeled off the transparent layer 120. The transparent layer 120 can exhibit wetting, which can displace gas to maintain contact with the platen 192. The transparent layer 120 can have a second side opposite the first side of the transparent layer 120.

The transparent layer 120 can be formed from silicone, silicone rubber, polymer rubber, or one or more other suitable materials.

The transparent layer 120 can have a surface area that corresponds to the surface area of the platen 192. For example, the transparent layer 120 can have a surface area that is slightly smaller than a surface area of platen 192, such that a user can easily position the various layers of the membrane 100 on the platen 192 with relatively loose tolerances. In some examples, the transparent layer 120 can have the same size and shape as the protective cover sheet layer 110. In other examples, the surface area of the transparent layer 120 can be slightly less than the surface area of the protective cover sheet layer 110, so that the protective cover sheet layer 110 extends slightly beyond a perimeter of the transparent layer 120. Such slight extension can protect the full surface area of the transparent layer 120 before the protective cover sheet layer 110 is peeled off the transparent layer 120.

The transparent layer 120 can be softer than the protective cover sheet layer 110. For example, the protective cover sheet layer 110 can protect the transparent layer 120 from being scratched or deformed by contact during handling of the membrane 100 or application of the transparent layer 120 to the platen 192. In addition, the protective cover sheet layer 110 can help protect the transparent layer 120 against contamination, which might otherwise become trapped between the transparent layer 120 and the platen 192 and disrupt the illumination and imaging of the anatomy.

The transparent layer 120, after being adhered to the platen 192, can be removable from the platen 192 by peeling the transparent layer 120 from the platen 192. For example, a user can work a corner of the transparent layer 120 away from the platen 192, then peel the transparent layer 120 away from the platen 192 to remove the remainder of the transparent layer 120. During typical use of the optical biometric sensor 190, the transparent layer 120 may be replaced after an extended period of time, such as once a day, once a week, once a month, or other suitable time frame.

The membrane 100 can include a stiffener layer 130 removably contacting the transparent layer 120 such that the stiffener layer 130 and the protective cover sheet layer 110 are disposed on opposite sides of the transparent layer 120. The transparent layer 120 and the stiffener layer 130 may be held together by surface tension, such as without using an additional adhesive material between the transparent layer 120 and the stiffener layer 130. The membrane 100 may optionally include a release agent disposed between the stiffener layer 130 and the transparent layer 120. The stiffener layer 130 can be removed, such as by being peeled off, the transparent layer 120 after the transparent layer 120 has been adhered to the platen 192. Alternatively, the stiffener layer 130 can be removed from the transparent layer 120 before the transparent layer 120 has been adhered to the platen 192.

The stiffener layer 130 can be formed from a clear polycarbonate film. The stiffener layer 130 can have a grade of graphics quality. The stiffener layer 130 can have a refractive index of 1.588. The stiffener layer 130 can have a light transmission of at least 88 percent. The stiffener layer 130 can have a hardness of Rockwell R 118. The stiffener layer 130 can have a yield strength of 8.5 pounds per square inch. The stiffener layer 130 can have an ultimate strength of 9 pounds per square inch. The stiffener layer 130 can have a thermal coefficient of expansion of 37.5 microinches per inch per degree Fahrenheit. The material and numerical properties are but mere examples; other materials and numerical values can also be used. During use of the biometric identification sensor, light does not interact with the stiffener layer 130 during use of the optical biometric sensor 190, using material that is at least partially transparent can allow for visual inspection of the membrane 100 during one or more stages of assembly of the membrane 100.

The stiffener layer 130 can be stiffer than the transparent layer 120. For example, the stiffener layer 130 can be sufficiently rigid to maintain a shape of the stiffener layer 130 as the membrane 100 is manufactured and handled.

The stiffener layer 130 can have a surface area that corresponds to the surface area of the platen 192. For example, the stiffener layer 130 can have a surface area that is slightly smaller than a surface area of platen 192, such that a user can easily position the various layers of the membrane 100 on the platen 192 with relatively loose tolerances. In some examples, the stiffener layer 130 can have the same size and shape as the transparent layer 120 and the protective cover sheet layer 110. Having the same size and shape in this manner can allow the membrane 100 to be manufactured as a sheet and cut into the suitable size and shape to correspond to the surface area of the platen 192. In other examples, the surface area of the stiffener layer 130 can be slightly greater than the surface area of the transparent layer 120, so that the stiffener layer 130 extends slightly beyond a perimeter of the transparent layer 120. Such slight extension can protect the full surface area of the stiffener layer 130 when the transparent layer 120 is being adhered to the platen 192.

The protective cover sheet layer 110 can be peeled off the transparent layer 120 when the transparent layer 120 is in contact with the stiffener layer 130, before the transparent layer 120 has been adhered to the platen 192.

In optical contact bonding, the transparent layer 120 can bond to the platen 192 without the use of any adhesive, such as by relying on friction, surface tension, and vacuum (because there may be no air between the platen 192 and the transparent layer 120). For example, the transparent layer 120 can be formed as a silicone rubber sheet. The transparent layer 120 can be embedded between the protective cover sheet layer 110 and the stiffener layer 130, each of which may optionally be formed from a plastic stiffening material (such as different plastic stiffening materials or the same plastic stiffening material). The stiffener layer 130 can reduce or eliminate distortion strain in the transparent layer 120 when the transparent layer 120 is applied onto the platen 192. The stiffener layer 130 can further maintain a flatness of the transparent layer 120 when the transparent layer 120 is applied onto the platen 192. For examples, the stiffener layer 130 can help apply the transparent layer 120 onto the platen 192 such that there are few or no air bubbles or gaps between the transparent layer 120 and the platen 192. The transparent layer 120 can be formed, within the membrane 100, to have optically flat surfaces. When the platen 192 is sufficiently clean or free from particles (such as by an optional cleaning step before the transparent layer 120 is applied onto the platen 192), the smooth surface of the platen 192 can experience a natural affinity to the corresponding smooth surface of the transparent layer 120. The corresponding smooth surfaces can form the optical contact simply by placing the transparent layer 120 in contact with the platen 192. The natural affinity between the transparent layer 120 and the platen 192 can create the optical contact almost entirely on its own, such as without the need to apply significant force to the stiffener layer 130 to force the transparent layer 120 against the platen 192.

In some configurations, the stiffener layer 130 and the protective cover sheet layer 110 can be formed from a same material, such as a clear polycarbonate material.

In some configurations, the stiffener layer 130 and the protective cover sheet layer 110 can be formed from a same material, have a same thickness, have a same stiffness, and have a same surface area that corresponds to a surface area of the platen 192. For these configurations, the membrane 100 can be symmetric, with the transparent layer 120 being disposed between layers that have a same or similar structure and function. Such a symmetric configuration can be beneficial for a user, in that either of the outer layers may be removed first, so that the user does not have to first determine which of the outer layers is the protective cover sheet layer 110 and which is the stiffener layer 130.

FIG. 2 shows a side-view schematic drawing of a portion of the membrane of FIG. 1, after the protective cover sheet layer 110 has been peeled off the transparent layer 120.

FIG. 3 shows a side-view schematic drawing of a portion of the membrane of FIG. 1, as the transparent layer 120 has been placed in contact with the platen 192 of the optical biometric sensor 190 to adhere the transparent layer 120 to the platen 192.

FIG. 4 shows a side-view schematic drawing of a portion of the membrane of FIG. 1, after the protective cover sheet layer 110 has been peeled off the transparent layer 120, after the transparent layer 120 has been adhered to the platen 192, and after the stiffener layer 130 has been peeled off the transparent layer 120. The optical biometric sensor 190 is ready for use.

FIG. 5 shows a side-view schematic drawing of an example of an optical biometric sensor 190 during use, in which the transparent layer 120 has been adhered to the platen 192.

The transparent layer 120, after being adhered to the platen 192, can receive an anatomy 180 of an individual. For example, during use of the optical biometric sensor 190, a person may place a finger or palm on the transparent layer 120, such that the transparent layer 120 is disposed between the finger or palm and the platen 192.

The optical biometric sensor 190 can illuminate, with sensor light 160, the anatomy 180 through the platen 192 and the transparent layer 120. The optical biometric sensor 190 can capture return light 170 returning from the anatomy 180 through the transparent layer 120 and the platen 192. For example, during use of the optical biometric sensor 190, a person may place a finger or palm on the transparent layer 120, such that the transparent layer 120 is disposed between the finger or palm and the platen 192.

The optical biometric sensor 190 can form an image of the anatomy 180 from the captured light. In some examples, the optical biometric sensor 190 can be part of a biometric identification system that can identify, enroll, and/or verify the individual based on the image of the anatomy 180. Other identification applications are also possible.

The optical biometric sensor 190 may illuminate the finger or palm through the platen 192 and the transparent layer 120. The optical biometric sensor 190 may capture light returning from the finger or palm through the transparent layer 120 and the platen 192 and may form an image of the fingerprint or palm print from the captured light. Because the illumination and return light passes through the transparent layer 120, the transparent layer 120 can be transparent at a wavelength of sensor light of the optical biometric sensor 190. Such transparency can help reduce or eliminate loss caused by absorption in the transparent layer 120.

The transparent layer 120 can have a refractive index that is within twenty percent of a refractive index of the platen 192. Matching the refractive indices of the transparent layer 120 and the platen 192, such as within twenty percent, can help reduce or eliminate reflections arising at the interface between the transparent layer 120 and the platen 192.

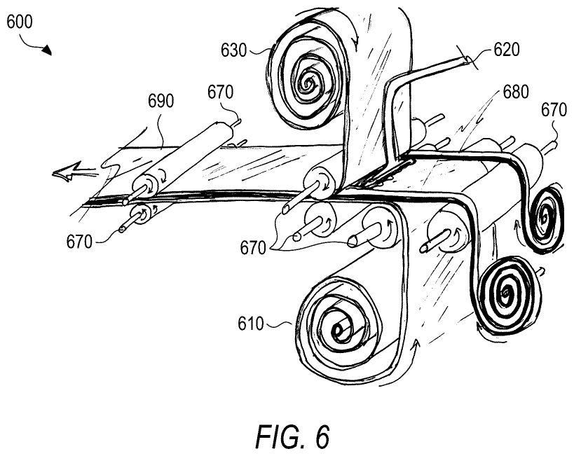

FIG. 6 shows a perspective-view drawing of an assembly apparatus 600 for assembling the membrane 100 of FIG. 1. A roll 610 of protective cover sheet layer material and a roll 630 of stiffener layer material each feed material through respective rollers 670 to emerge as opposing layers of an uncut membrane sheet. The rollers 670 can compress the layers and uncut membrane sheet to specified thicknesses with relatively loose tolerances, which is suitable for the protective cover sheet layer 110 and the stiffener layer 130. Because the transparent layer 120 may be part of the optical path of the optical biometric sensor 190, the transparent layer 120 may have a tighter thickness tolerance than the protective cover sheet layer 110 and the stiffener layer 130. In addition, the transparent layer material 620 may not have enough structural integrity to be formed into a sheet or a roll. For at least these reasons, transparent layer material 620 may be provided as a liquid. The liquid may be dispensed between two strips of spacer material 680, on top of one of the other uncut layers, such as the uncut protective cover sheet layer material (as shown in FIG. 6) or the uncut stiffener layer material. The spacer material 680 can have a thickness that matches a desired thickness for the transparent layer 120. Rollers 670 can force the layers of the uncut membrane together to form a membrane sheet 690. The spacer material 680 can be relatively incompressible, so that the rollers 670 may not significantly change the thickness of the spacer material 680. The roller 670 can direct the membrane sheet 690 to a subsequent curing station, which can solidify the liquid to form the transparent layer 120. The roller 670 can direct the cured membrane sheet 690 to a subsequent cutting station, which can cut the cured membrane sheet 690 to a suitable size and shape. The cutting station can cut the membrane sheet 690 so that the spacer material 680 may be excluded from the membrane 100.

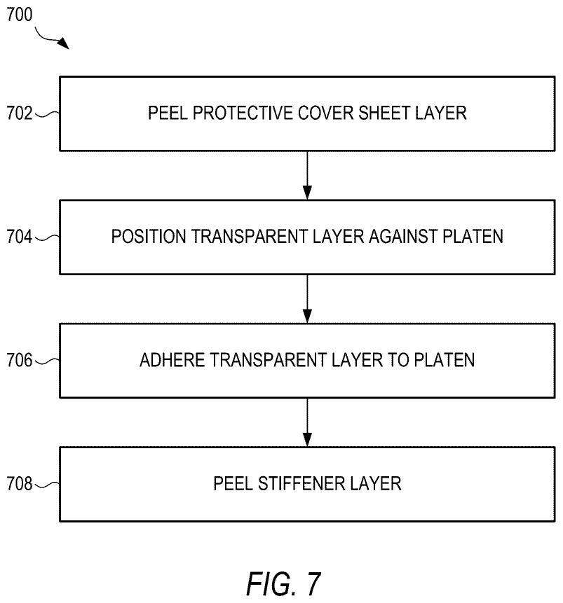

FIG. 7 shows a flow chart of an example of a method for removably adhering a transparent layer, such as transparent layer 120, of a membrane, such as membrane 100, to a platen, such as platen 192, of an optical biometric sensor, such as optical biometric sensor 190. The membrane includes the transparent layer disposed between a protective cover sheet layer, such as protective cover sheet layer 110, and a stiffener layer, such as stiffener layer 130. The method 700 can be executed by an operator of the optical biometric sensor, such as a member of a security team. While possible, the method 700 may not typically be executed by the individuals being scanned for identification. The method 700 is but one example of a method for removably adhering a membrane to a platen of an optical biometric sensor; other methods can also be used.

At operation 702, the method 700 can include peeling the protective cover sheet layer from the transparent layer to expose a first side of the transparent layer.

At operation 704, the method 700 can include positioning the first side of the transparent layer against the platen such that the transparent layer is disposed between the platen and the stiffener layer.

At operation 706, the method 700 can include adhering the transparent layer to the platen via optical contact bonding.

At operation 708, the method 700 can include peeling the stiffener layer from the transparent layer after the transparent layer has been adhered to the platen to expose a second side, opposite the first side, of the transparent layer.

The second side of the transparent layer can, after the transparent layer has been adhered to the platen, receive an anatomy of a user for forensic analysis, enrolling, verifying, or identifying the user via the optical biometric sensor.

The transparent layer, after being adhered to the platen, can be removable from the platen by peeling the transparent layer from the platen.

The stiffener layer can be stiffer than the transparent layer and stiffer than the protective cover sheet layer.

The transparent layer can be softer than the protective cover sheet layer.

The stiffener layer and the protective cover sheet layer can be formed from a same material, can have a same thickness, can have a same stiffness, and can have a same surface area that corresponds to a surface area of the platen.

The protective cover sheet layer, the transparent layer, and the stiffener layer each have respective surface areas that corresponds to a surface area of the platen.

The transparent layer can be transparent at a wavelength of sensor light of the optical biometric sensor.

The transparent layer can have a refractive index that is within twenty percent of a refractive index of the platen.

To further illustrate the system and method disclosed herein, a non-limiting list of examples is provided below. Each of the following non limiting examples can stand on its own or can be combined in any permutation or combination with any one or more of the other examples.

In Example 1, a membrane for a platen of an optical biometric sensor, the membrane can comprise: a protective cover sheet layer; a transparent layer removably contacting the protective cover sheet layer and configured to adhere, via optical contact bonding, to the platen after the protective cover sheet layer has been removed; and a stiffener layer removably contacting the transparent layer such that the stiffener layer and the protective cover sheet layer are disposed on opposite sides of the transparent layer, the stiffener layer being removable from the transparent layer after the transparent layer has been adhered, via optical contact bonding, to the platen.

In Example 2, the membrane of Example 1 can optionally be configured such that the protective cover sheet layer is configured to be peeled off the transparent layer when the transparent layer is in contact with the stiffener layer, before the transparent layer has been adhered to the platen.

In Example 3, the membrane of any one of Examples 1-2 can optionally be configured such that the stiffener layer is configured to be peeled off the transparent layer after the transparent layer has been adhered to the platen.

In Example 4, the membrane of any one of Examples 1-3 can optionally be configured such that the transparent layer, after being adhered to the platen, is configured to receive an anatomy of a user for forensic analysis, enrolling, verifying, or identifying the user via the optical biometric sensor.

In Example 5, the membrane of any one of Examples 1˜4 can optionally be configured such that the transparent layer, after being adhered to the platen, is removable from the platen by peeling the transparent layer from the platen.

In Example 6, the membrane of any one of Examples 1-5 can optionally be configured such that the stiffener layer is stiffer than the transparent layer.

In Example 7, the membrane of any one of Examples 1-6 can optionally be configured such that the transparent layer is softer than the protective cover sheet layer.

In Example 8, the membrane of any one of Examples 1-7 can optionally be configured such that the protective cover sheet layer, the transparent layer, and the stiffener layer each have respective surface areas that corresponds to a surface area of the platen.

In Example 9, the membrane of any one of Examples 1-8 can optionally be configured such that the stiffener layer and the protective cover sheet layer are formed from a same material.

In Example 10, the membrane of any one of Examples 1-9 can optionally be configured such that the stiffener layer and the protective cover sheet layer are formed from a same material, have a same thickness, have a same stiffness, and have a same surface area that corresponds to a surface area of the platen.

In Example 11, the membrane of any one of Examples 1-10 can optionally be configured such that the transparent layer is transparent at a wavelength of sensor light of the optical biometric sensor.

In Example 12, the membrane of any one of Examples 1-11 can optionally be configured such that the transparent layer has a refractive index that is within twenty percent of a refractive index of the platen.

In Example 13, a method for removably adhering a transparent layer of a membrane to a platen of an optical biometric sensor, the membrane comprising the transparent layer disposed between a protective cover sheet layer and a stiffener layer, can comprise: peeling the protective cover sheet layer from the transparent layer to expose a first side of the transparent layer; positioning the first side of the transparent layer against the platen such that the transparent layer is disposed between the platen and the stiffener layer; adhering the transparent layer to the platen via optical contact bonding; and peeling the stiffener layer from the transparent layer after the transparent layer has been adhered to the platen to expose a second side, opposite the first side, of the transparent layer.

In Example 14, the method of Example 13 can optionally be configured such that the second side of the transparent layer is configured to, after the transparent layer has been adhered to the platen, receive an anatomy of a user for forensic analysis, enrolling, verifying, or identifying the user via the optical biometric sensor.

In Example 15, the method of any one of Examples 13-14 can optionally be configured such that the transparent layer, after being adhered to the platen, is removable from the platen by peeling the transparent layer from the platen.

In Example 16, the method of any one of Examples 13-15 can optionally be configured such that: the stiffener layer is stiffer than the transparent layer and stiffer than the protective cover sheet layer; and the transparent layer is softer than the protective cover sheet layer.

In Example 17, the method of any one of Examples 13-16 can optionally be configured such that the stiffener layer and the protective cover sheet layer are formed from a same material, have a same thickness, have a same stiffness, and have a same surface area that corresponds to a surface area of the platen.

In Example 18, the method of any one of Examples 13-17 can optionally be configured such that: the transparent layer is transparent at a wavelength of sensor light of the optical biometric sensor; and the transparent layer has a refractive index that is within twenty percent of a refractive index of the platen.

In Example 19, a membrane for a platen of an optical biometric sensor can comprise: a protective cover sheet layer having a surface area that corresponds to a surface area of the platen; a transparent layer having a first side that removably contacts the protective cover sheet layer such that the protective cover sheet layer can be peeled off the transparent layer, the first side of the transparent layer being configured to adhere, via optical contact bonding, to the platen after the protective cover sheet layer has been peeled off the transparent layer by being placed in contact with the platen, the transparent layer being softer than the protective cover sheet layer, the transparent layer having a surface area that corresponds to the surface area of the platen, the transparent layer having a second side opposite the first side of the transparent layer; and a stiffener layer having a first side that removably contacts the second side of the transparent layer, the stiffener layer being stiffer than the transparent layer, the stiffener layer and the protective cover sheet layer being formed from a same material, having a same thickness, having a same stiffness, and having a same surface area that corresponds to the surface area of the platen, the stiffener layer being configured to be peeled off the transparent layer after the transparent layer has been adhered to the platen to expose the second side of the transparent layer.

In Example 20, the membrane of Example 19 can optionally be configured such that: the transparent layer is transparent at a wavelength of sensor light of the optical biometric sensor; and the transparent layer has a refractive index that is within twenty percent of a refractive index of the platen.

1. A membrane for a platen of an optical biometric sensor, the membrane comprising:
a protective cover sheet layer;
a transparent layer removably contacting the protective cover sheet layer and configured to adhere, via optical contact bonding, to the platen after the protective cover sheet layer has been removed; and
a stiffener layer removably contacting the transparent layer such that the stiffener layer and the protective cover sheet layer are disposed on opposite sides of the transparent layer, the stiffener layer being removable from the transparent layer after the transparent layer has been adhered, via optical contact bonding, to the platen.

2. The membrane of claim 1, wherein the protective cover sheet layer is configured to be peeled off the transparent layer when the transparent layer is in contact with the stiffener layer, before the transparent layer has been adhered to the platen.

3. The membrane of claim 1, wherein the stiffener layer is configured to be peeled off the transparent layer after the transparent layer has been adhered to the platen.

4. The membrane of claim 1, wherein the transparent layer, after being adhered to the platen, is configured to receive an anatomy of a user for forensic analysis, enrolling, verifying, or identifying the user via the optical biometric sensor.

5. The membrane of claim 1, wherein the transparent layer, after being adhered to the platen, is removable from the platen by peeling the transparent layer from the platen.

6. The membrane of claim 1, wherein the stiffener layer is stiffer than the transparent layer.

7. The membrane of claim 1, wherein the transparent layer is softer than the protective cover sheet layer.

8. The membrane of claim 1, wherein the protective cover sheet layer, the transparent layer, and the stiffener layer each have respective surface areas that corresponds to a surface area of the platen.

9. The membrane of claim 1, wherein the stiffener layer and the protective cover sheet layer are formed from a same material.

10. The membrane of claim 1, wherein the stiffener layer and the protective cover sheet layer are formed from a same material, have a same thickness, have a same stiffness, and have a same surface area that corresponds to a surface area of the platen.

11. The membrane of claim 1, wherein the transparent layer is transparent at a wavelength of sensor light of the optical biometric sensor.

12. The membrane of claim 1, wherein the transparent layer has a refractive index that is within twenty percent of a refractive index of the platen.

13. A method for removably adhering a transparent layer of a membrane to a platen of an optical biometric sensor, the membrane comprising the transparent layer disposed between a protective cover sheet layer and a stiffener layer, the method comprising:
peeling the protective cover sheet layer from the transparent layer to expose a first side of the transparent layer;
positioning the first side of the transparent layer against the platen such that the transparent layer is disposed between the platen and the stiffener layer;
adhering the transparent layer to the platen via optical contact bonding; and
peeling the stiffener layer from the transparent layer after the transparent layer has been adhered to the platen to expose a second side, opposite the first side, of the transparent layer.

14. The method of claim 13, wherein the second side of the transparent layer is configured to, after the transparent layer has been adhered to the platen, receive an anatomy of a user for forensic analysis, enrolling, verifying, or identifying the user via the optical biometric sensor.

15. The method of claim 13, wherein the transparent layer, after being adhered to the platen, is removable from the platen by peeling the transparent layer from the platen.

16. The method of claim 13, wherein:
the stiffener layer is stiffer than the transparent layer and stiffer than the protective cover sheet layer; and
the transparent layer is softer than the protective cover sheet layer.

17. The method of claim 13, wherein the stiffener layer and the protective cover sheet layer are formed from a same material, have a same thickness, have a same stiffness, and have a same surface area that corresponds to a surface area of the platen.

18. The method of claim 13, wherein:
the transparent layer is transparent at a wavelength of sensor light of the optical biometric sensor; and
the transparent layer has a refractive index that is within twenty percent of a refractive index of the platen.

19. A membrane for a platen of an optical biometric sensor, the membrane comprising:
a protective cover sheet layer having a surface area that corresponds to a surface area of the platen;
a transparent layer having a first side that removably contacts the protective cover sheet layer such that the protective cover sheet layer can be peeled off the transparent layer, the first side of the transparent layer being configured to adhere, via optical contact bonding, to the platen after the protective cover sheet layer has been peeled off the transparent layer by being placed in contact with the platen,
the transparent layer being softer than the protective cover sheet layer,
the transparent layer having a surface area that corresponds to the surface area of the platen,
the transparent layer having a second side opposite the first side of the transparent layer; and
a stiffener layer having a first side that removably contacts the second side of the transparent layer,
the stiffener layer being stiffer than the transparent layer,
the stiffener layer and the protective cover sheet layer being formed from a same material, having a same thickness, having a same stiffness, and having a same surface area that corresponds to the surface area of the platen,
the stiffener layer being configured to be peeled off the transparent layer after the transparent layer has been adhered to the platen to expose the second side of the transparent layer.

20. The membrane of claim 19, wherein:
the transparent layer is transparent at a wavelength of sensor light of the optical biometric sensor, and
the transparent layer has a refractive index that is within twenty percent of a refractive index of the platen.
